(19) United States
(12) Patent Application Publication
(10) Pub. No.: US 2011/0201804 A1
(43) Pub. Date: Aug. 18, 2011

(54) PROCESS FOR THE PREPARATION OF 1- ( 3-HYDROXYMETHYLPYRID-2 -YL ) -2 -PHENYL-4-METHYLPIPERAZINE AND MIRTAZAPINE

(75) Inventors: Manjunath Narayan Bhanu, Pune (IN); Samir Naik, Thane (IN); Prashant Joshi, Kalyan (IN); Vijay Sharma, Mumbia (IN)

(73) Assignee: WATSON PHARMA PRIVATE LIMITED, Andheri (E), Mumbai (IN)

(21) Appl. No.: 13/063,085

(22) PCT Filed: Oct. 20, 2009
(86) PCT No.: PCT/IB2009/054625

§ 371 Date: Mar. 9, 2011

(30) Foreign Application Priority Data
Oct. 22, 2008 (IN) 2279/MUM/2008

(51) Int. Cl. C07D 487/14; C07D 401/04

(52) U.S. Cl. 540/578; 544/360

(57) ABSTRACT

Disclosed herein is a process for the manufacture of mirtazapine and intermediates useful in preparing mirtazapine which includes the reduction of 1-(3-carboxypyridyl-2)-4-methyl-2-phenylpiperazine with an organoaluminum hydride.

### FIELD OF THE INVENTION

The present invention relates to an improved process for preparing mirtazapine and its intermediate 1-(3-hydroxymethylpyridyl-2)-4-methyl-2-phenyl piperazine, a key intermediate used in the manufacture of mirtazapine.

### BACKGROUND OF THE INVENTION

The present invention relates to 1,2,3,4,10,14b-hexahydro-2-methylpyrazino[2,1-a]pyrido[2,3-c][2]benzazepine, also known as mirtazapine (Formula I)

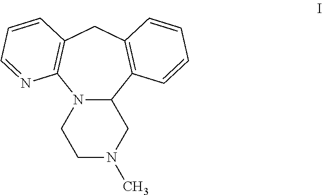

Mirtazapine has a tetracyclic chemical structure unrelated to other classes of antidepressants such as selective serotonin reuptake inhibitors, tricyclics or monoamine oxidase inhibitors. Mirtazapine belongs to the piperazinoazepine group of compounds. Mirtazapine is sold under the trademark REMERON® and is available in two dosage forms: tablets and orally disintegrating tablets. Both dosage forms of REMERON® are indicated for the treatment of major depressive disorder.

Mirtazapine acts as an antagonist at central presynaptic α₂-adrenergic autoreceptors and heteroreceptors, thereby possibly resulting in increased central noradrenergic and serotonergic neurotransmission. Mirtazapine is a potent antagonist of serotonin type 2 (5-HT₂) and type 3 (5-HT₃) receptors, but the drug does not exhibit any significant affinity for serotonin type 1A (5-HT_{1A}) or type 1B (5-HT_{1B}) receptors. Mirtazapine is a potent antagonist of histamine (H₁) receptors, is a moderate antagonist at muscarinic receptors and exhibits moderate peripheral α₂-adrenergic blocking activity. Because of its unique pharmacodynamic properties, mirtazapine is an effective, safe and well-tolerated antidepressant agent.

Mirtazapine can be manufactured by methods described in U.S. Pat. No. 4,002,848 (“the '848 patent”) (assigned to Akzona Incorporated). The '848 patent discloses a process for preparing mirtazapine by adding concentrated sulfuric acid to 1-(3-hydroxymethylpyridyl-2)-2-phenyl-4-methylpiperazine of Formula II.

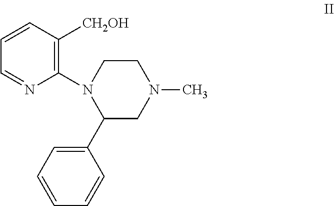

1-(3-hydroxymethylpyridyl-2)-2-phenyl-4-methylpiperazine of Formula II, also known as a pyridinemethanol compound, is a key raw material for the manufacture of mirtazapine.

The '848 patent describes a process for preparation of 1-(3-hydroxymethylpyridyl-2)-2-phenyl-4-methylpiperazine by reducing a pyridinecarboxylic acid of Formula III

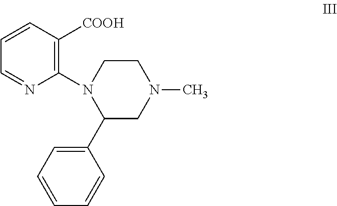

using lithium aluminum hydride.

European Published Patent Application No. 1 238 977 discloses a process for preparation of 1-(3-hydroxymethylpyridyl-2)-2-phenyl-4-methylpiperazine by reducing the potassium pyridinecarboxylate represented by Formula IV:

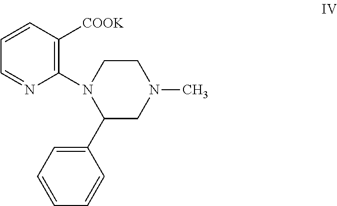

using lithium aluminum hydride.

Both of the above processes use lithium aluminum hydride as a reducing agent, which is a pyrophoric substance and a potential hazard in the scale-up and commercial manufacturing of 1-(3-hydroxymethylpyridyl-2)-2-phenyl-4-methylpiperazine.

U.S. Patent Publication No. 2003/10069417 discloses a process for the preparation of 1-(3-hydroxymethylpyridyl-2)-2-phenyl-4-methylpiperazine by reacting 2-amino-3-hydroxymethylpyridine with N-methyl-1-phenyl-2,2′-iminodiethyl chloride. However, the process is economically infeasible because of the expensive raw materials.

Since the above processes have various drawbacks, there is a need for a process useful for the preparation of 1-(3-hydroxymethylpyridyl-2)-2-phenyl-4-methylpiperazine which is commercially scalable, non-hazardous and can be commercially viable.

### SUMMARY OF THE INVENTION

The present invention relates to a process for preparing 1-(3-hydroxymethylpyridyl-2)-2-phenyl-4-methylpiperazine which is a key raw material in the manufacture of mirtazapine.

The present invention also relates to a process for preparing mirtazapine.

The present invention further provides a process for preparing 1-(3-hydroxymethylpyridyl-2)-2-phenyl-4-methylpiperazine which is scalable, free from hazardous pyrophoric chemicals and commercially viable.

The process for preparing 1-(3-hydroxymethylpyridyl-2)-2-phenyl-4-methylpiperazine according to the present invention comprises the step of reacting a pyridinecarboxylate, preferably a pyridinecarboxylate metal salt or pyridinecarboxylic acid with an organoaluminum hydride such as sodium bis(2-methoxyethoxy) aluminum hydride.

The process for preparing mirtazapine according to the present invention comprises the step of reacting a pyridinecarboxylate, preferably a pyridinecarboxylate metal salt or pyridinecarboxylic acid with an organoaluminum hydride such as sodium bis(2-methoxyethoxy) aluminum hydride.

On embodiment of the present invention for preparing mirtazapine further comprises the steps of:
a) hydrolyzing 1-(3-cyanopyridyl-2)-4-methyl-2-phenylpiperazine to give 1-(3-carboxypyridyl-2)-4-methyl-2-phenylpiperazine of Formula III;
(b) reducing 1-(3-carboxypyridyl-2)-4-methyl-2-phenylpiperazine of Formula III with an organoaluminum hydride such as sodium bis(2-methoxyethoxy)aluminum hydride to give 1(3-hydroxymethylpyridyl-2)-2-phenyl-4-methylpiperazine of Formula II; and
(c) cyclizing the 1-(3-hydroxymethylpyridyl-2)-2-phenyl-4-methylpiperazine of Formula II to give mirtazapine of Formula I.

The above processes are non-hazardous since they avoid the use of highly reactive and flammable reducing reagents such as lithium aluminum hydride.

### DESCRIPTION OF THE INVENTION

1-(3-hydroxymethylpyridyl-2)-2-phenyl-4-methylpiperazine, sometimes referred to as a pyridinemethanol, is a compound that is useful in the production of mirtazapine. The present invention relates to a method for preparing 1-(3-hydroxymethylpyridyl-2)-2-phenyl-4-methylpiperazine which involves reacting a pyridinecarboxylate with an organoaluminum hydride, preferably an organoaluminum hydride that is not highly reactive and readily flammable with air or water.

One embodiment of the present invention for preparing 1-(3-hydroxymethylpyridyl-2)-2-phenyl-4-methylpiperazine comprises the following steps:
(a) suspending a pyridinecarboxylate such as a pyridinecarboxylate metal salt or a pyridinecarboxylic acid in an organic solvent, preferably an aromatic solvent such as toluene;
(b) adding an organoaluminum hydride such as sodium bis(2-methoxyethoxy)aluminum hydride as a reducing agent to the reaction mixture of step (a) to produce 1-(3-hydroxymethylpyridyl-2)-2-phenyl-4-methylpiperazine;
(c) quenching the mixture of step (b), preferably once the reduction of the pyridinecarboxylate has been completed or substantially completed; and
(d) isolating the 1-(3-hydroxymethylpyridyl-2)-2-phenyl-4-methylpiperazine from the reaction mass.

The organoaluminum hydride employed in the above process may be used in a molar excess to pyridinecarboxylate. For example, molar ratio of organoaluminum hydride to pyridinecarboxylate should be about 2 moles of organoaluminum hydride per mole of pyridinecarboxylate to about 6 moles of organoaluminum hydride per mole of pyridinecarboxylate, preferably about 2.5 moles of organoaluminum hydride per mole of pyridinecarboxylate to about 5 moles of organoaluminum hydride per mole of pyridinecarboxylate and most preferably about 3 moles of organoaluminum hydride per mole of pyridinecarboxylate to about 3.5 moles of organoaluminum hydride per mole of pyridinecarboxylate.

In one embodiment of the present invention, the organoaluminum hydride is dissolved or suspended in an organic solvent, preferably an aromatic solvent, prior to the addition to the pyridinecarboxylate suspension. Although not necessary, it is preferred that the organic solvent for suspending the pyridinecarboxylate and the organic solvent for dissolving or suspending the organoaluminum hydride be the same organic solvent, preferably the same aromatic solvent such as toluene. The reduction reaction between the pyridinecarboxylate and the organoaluminum halide may be conducted at a temperature of about 10° C. to about 50° C., preferably about 15° C. to about 40° C. and most preferably about 20° C. to about 35° C. The reduction reaction may also occur at a time period of about 2 to about 10 hours, preferably about 3 to about 8 hours and most preferably about 4 to about 6 hours. The time and temperature of the reduction reaction may vary depending upon the concentration and amounts of the reactants and solvents employed in the reaction.

The quenching step during the production of 1-(3-hydroxymethyllpyridyl-2)-2-phenyl-4-methylpiperazine is conducted once all or substantially all the pyridinecarboxylate has been reduced to 1-(3-hydroxymethyllpyridyl-2)-2-phenyl-4-methylpiperazine. The quenching step may occur about 4 to about 6 hours after the reactants are combined in a molar ratio of about 3 to about 3.5 moles of organoaluminum halide per mole of pyridinecarboxylate. The quenching step may be conducted by any means that cause the organoaluminum hydride to decompose.

In one embodiment of the present invention, the quenching step is conducted by adding an excess volume of an organic solvent such as a C₁ to C₄ alcohol, and an excess volume of an aqueous solution of an alkali or alkali earth metal salt such as sodium sulfate to the reduction reaction mass. The volume of organic solvent added during the quenching step should be should be at least 1.5 times, preferably at least 2 times, the volume of organic solvent originally used to suspend the pyridinecarboxylate. The volume of the aqueous salt solution added during the quenching step should be at least 1.5 times, preferably at least 2 times, and most preferably at least 3 times, the volume of organic solvent originally used to suspend the pyridinecarboxylate.

The concentration of the alkali or alkali earth metal salt in the aqueous salt solution employed in the quenching step may be about 20% to about 75% w/v of the aqueous salt solution, preferably about 30% to about 60% w/v and most preferably about 45% to about 55% w/v.

The quenching materials should be selected so the organoaluminum hydride is decomposed into a granular solid that may be easily filtered and separated from the quenched reaction mixture.

The resulting 1-(3-hydroxymethyllpyridyl-2)-2-phenyl-4-methylpiperazine should be isolated from the quenched reaction mixture. The isolated product can be in the form or a solution, suspension, slurry or crystal. The isolated product may be subsequently converted to mirtazapine.

Mirtazapine can be prepared from the isolated 1-(3-hydroxymethyllpyridyl-2)-2-phenyl-4-methylpiperazine by employing a ring closure or cyclization reaction. In one embodiment of the present invention, the ring closure or cyclization of the isolated 1-(3-hydroxymethyllpyridyl-2)-2-phenyl-4-methylpiperazine is performed by combining the 1-(3-hydroxymethyllpyridyl-2)-2-phenyl-4-methylpiperazine with a dehydrating agent. Suitable dehydrating agents include acids such as sulfuric acid, hydrochloric acid, trifluoroacetic acid, phosphoric acid, polyphosphoric acid, phosphorous oxychloride, phosphorous trioxide and phosphorous pentoxide.

The present invention also includes a process for preparing mirtazapine that comprises the following steps:
(a) hydrolyzing the compound 1-(3-cyanopyridyl-2)-4-methyl-2-phenylpiperazine to form 1-(3-carboxypyridyl-2)-4-methyl-2-phenylpiperazine;
(b) reducing the 1-(3-carboxypyridyl-2)-4-methyl-2-phenylpiperazine with an organoaluminum hydride such as sodium bis(2-methoxyethoxy)aluminum to give 1-(3-hydroxymethyllpyridyl-2)-2-phenyl-4-methylpiperazine; and
(c) cyclizating 1-(3-hydroxymethyllpyridyl-2)-2-phenyl-4-methylpiperazine with a dehydrating agent to give mirtazapine.

In one embodiment of the present invention, the hydrolyzing step in the production of mirtazapine comprises dissolving or suspending 1-(3-cyanopyridyl-2)-4-methyl-2-phenylpiperazine in one or more suitable solvents selected from the group consisting of water, polar aprotic solvents, C₁ to C₄ alcohols, or mixtures thereof. Examples of useful polar aprotic solvents include dimethylformamide, dimethylacetamide and dimethylsulfoxide. Examples of useful alcohols include methanol, ethanol, propanol, isopropanol and butanol.

Once the 1-(3-cyanopyridyl-2)-4-methyl-2-phenylpiperazine is dissolved or suspended, it is reacted with a suitable amount of base, preferably an alkali metal base such as potassium hydroxide, sodium hydroxide, or mixtures thereof, in a molar ratio ranging from about 10 moles of base per mole of 1-(3-cyanopyridyl-2)-4-methyl-2-phenylpiperazine to about 30 moles of alkali base per mole of 1-(3-cyanopyridyl-2)-4-methyl-2-phenylpiperazine, preferably about 15 moles of base per mole of 1-(3-cyanopyridyl-2)-4-methyl-2-phenylpiperazine to about 25 moles of base per mole of 1-(3-cyanopyridyl-2)-4-methyl-2-phenylpiperazine and most preferably about 18 moles of base per mole of 1-(3-cyanopyridyl-2)-4-methyl-2-phenylpiperazine to about 22 moles of base per mole of 1-(3-cyanopyridyl-2)-4-methyl-2-phenylpiperazine.

The hydrolysis reaction may be conducted by heating the reaction mass to a temperature range between about 60° C. to about 95° C., preferably between about 80° C. to about 90° C. and most preferably about 85° C. to about 88° C. for about 5 to about 10 hours, more preferably between about 6 to about 8 hours to provide 1-(3-carboxypyridyl-2)-4-methyl-2-phenylpiperazine. Once the hydrolysis reaction is completed, the 1-(3-carboxypyridyl-2)-4-methyl-2-phenylpiperazine will be isolated from the reaction mass by conventional procedures, an example of which is provided below in Example 1.

In one embodiment of the present invention, the reduction step in the production of mirtazapine comprises reacting 1-(3-carboxypyridyl-2)-4-methyl-2-phenylpiperazine with an organoaluminum hydride such as sodium bis(2-methoxyethoxy)aluminum to give 1-(3-hydroxymethyllpyridyl-2)-2-phenyl-4-methylpiperazine. The reduction step is described in detail above and may include the following steps:
(a) suspending 1-(3-carboxypyridyl-2)-4-methyl-2-phenylpiperazine in an organic solvent, preferably an aromatic solvent such as toluene;
(b) adding an organoaluminum hydride solution such as a solution of sodium bis(2-methoxyethoxy)aluminum hydride in an organic solvent, preferably an aromatic solvent such as toluene to the suspension of step (a) to form 1-(3-hydroxymethyllpyridyl-2)-2-phenyl-4-methylpiperazine;
(c) quenching the mixture of step (b), preferably once the reduction of 1-(3-carboxypyridyl-2)-4-methyl-2-phenylpiperazine has been completed or substantially completed; and
(d) isolating the 1-(3-hydroxymethyllpyridyl-2)-2-phenyl-4-methylpiperazine.

Once the 1-(3-hydroxymethyllpyridyl-2)-2-phenyl-4-methylpiperazine is isolated, it can be converted to mirtazapine by the ring closure or cyclization processes previously described. One embodiment of the ring closure process comprises reacting 1-(3-hydroxymethyllpyridyl-2)-2-phenyl-4-methylpiperazine with sulfuric acid, preferably concentrated sulfuric acid. In this embodiment, the temperature of the reaction mass is maintained between about 15° C. to about 45° C., preferably about 20° C. to about 40° C., and most preferably about 25° C. to about 30° C. After stirring the reaction mass for a suitable amount of time, typically about 5 to 20 hours, the pH of the reaction mass is adjusted to about 9-12, preferably about 10-11, using a suitable base such as a 20-25% aqueous ammonium solution. The temperature of the reaction mass during the pH adjustment should be kept below 40° C., preferably below 35° C., and most preferably below 30° C. After the pH adjustment, the mirtazapine is isolated from the reaction mass by conventional techniques such as those described below in Example 3. Additional isolation methods can be found in U.S. Pat. No. 4,062,848; European Published Patent Application No. 1 238 977; and United States Published Patent Application 2003/0069417, which are incorporated herein by reference.

### EXAMPLES

### Example 1

### Preparation of 1-(3-carboxypyridyl-2)-4-methyl-2-phenylpiperazine

175 grams (0.63 mole) of 1-(3-cyanopyridyl-2)-4-methyl-2-phenylpiperazine was dissolved in 1150 ml methanol. 650 grams (11.6 mole) of potassium hydroxide was slowly added to the reaction mixture under stirring to obtain a clear solution. 250 ml of water was added and the reaction mixture was heated to reflux at 85° C. to 90° C. The reaction mixture was maintained under reflux for 24 hours. The reaction mixture was then cooled to 50° C. The reaction mixture was concentrated under vacuum to remove the methanol. The thick brown mass obtained was then cooled to about 25° C. and 350 ml of water was added, and the reaction mass was stirred for 15 minutes. The pH of the reaction mass was adjusted to about 7 using concentrated hydrochloric acid and maintained at a temperature between 20° C. to 30° C. The resulting suspension obtained was stirred for 30 minutes at 20° C. to 30° C. The solids were filtered and dried at 60° C. for 4 to 5 hours. The dried solids were then suspended in 475 ml of isopropyl alcohol and the reaction mixture was heated to reflux and stirred at reflux temperature for about 30 minutes. The reaction mixture was filtered hot to remove the insoluble inorganics. The clear filtrate was then concentrated under vacuum to get a thick slurry. The resulting slurry was then cooled to about 10° C. and filtered. The product was washed with 100 ml n-hexane. The wet product was dried at 60° C.-70° C. to get 90 grams of 1-(3-carboxypyridyl-2)-4-methyl-2-phenylpiperazine.

### Example 2

### Preparation of 1-(3-hydroxymethylpyridyl-2)-2-phenyl-4-methylpiperazine

50 grams (0.168 mole) of 1-(3-carboxypyridyl-2)-4-methyl-2 phenylpiperazine was suspended in 500 ml anhydrous toluene. The reaction mixture was cooled to 15° C. 178 ml of a toluene solution of sodium bis(2-methoxyethoxy)aluminum hydride, also known as VITRIDE®, (65% solution in toluene) was slowly added to the reaction mixture maintaining the temperature between 15° C. to 20° C. in about 1 to 2 hours. The temperature of the reaction mixture was then raised to 25° C. and the reaction mixture was stirred for 5 hours at 25° C. to 30° C. The reaction mixture was cooled to 10° C. and 100 ml of methanol was slowly added to the reaction mixture maintaining temperature below 30° C. The reaction mass was further stirred at 25° C. to 30° C. for 1 hour. 150 ml aqueous solution of sodium sulfate (50%) was added to the reaction mixture at about 30° C. The reaction mixture was then heated to about 60° C. and stirred for 1 hour. The reaction mass was filtered to remove the inorganic materials obtained. The inorganic materials were washed with 200 ml toluene. The clear filtrate and the washings were collected together and the layers were separated. The upper toluene extract was then washed with 200 ml water. The toluene extract was dried over anhydrous sodium sulfate and then concentrated under vacuum maintaining temperature below 65° C. until a thick slurry was obtained. 100 ml of hexane was added and the reaction mass was cooled to 10° C. The product obtained was filtered and washed with 25 ml hexane. The wet solids were dried at about 60° C. to 65° C. to get 40 grams of 1-(3-hydroxymethylpyridyl-2)-2-phenyl-4-methylpiperazine.

### Example 3

### Preparation of mirtazapine (I) from 1-(3-hydroxymethylpyridyl-2)-2-phenyl-4-methylpiperazine

100 ml of concentrated sulfuric acid was cooled to about 15° C. 50 grams (0.18 mole) of 1-(3-hydroxymethylpyridyl-2)-2-phenyl-4-methylpiperazine was added slowly to the sulfuric acid, and the temperature was maintained below 20° C. The temperature was then raised to 30° C., and the reaction mixture was stirred for 12 hours maintaining the temperature between 25° C. to 30° C. The reaction mass was then quenched in 1 liter of ice cold water. The pH of the reaction mixture was adjusted to about 10-11 using 20% to 25% aqueous ammonia solution while maintaining the temperature below 30° C. 500 ml of ethylacetate was added to the reaction mixture and stirred for about 15 minutes at 30° C. The layers were separated, and the aqueous layer was back extracted with 100 ml of ethylacetate. All the ethylacetate extracts were combined together and heated to reflux under stirring. 5 grams of activated charcoal was added, and the reaction mixture was stirred under reflux temperature for 30 minutes. The reaction mixture was filtered hot over a hyflo bed. 1.6 ml of water was added to the clear filtrate and heated to about 50° C. The reaction mass was stirred at 50° C. for 30 minutes and then concentrated under vacuum to keep about 100 ml of ethylacetate in the reaction mixture. 150 ml of isopropyl ether was added to the reaction mass and heated to reflux. 5 grams of activated carbon was added, and the reaction mixture was stirred under reflux for 30 minutes. The reaction mixture was filtered hot over a hyflo bed. The clear filtrate was cooled under stirring to about 30° C. and further chilled to about 5° C. The reaction mass was stirred at 5° C. to 10° C. for 1 hour. The resulting solid crystals were filtered and washed with 25 ml of chilled isopropyl ether. The product obtained was suck dried for 30 minutes and then dried at 65° C. under vacuum to get 40 grams of mirtazapine having HPLC purity of more than 99.8%.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A process for preparing 1-(3-hydroxymethylpyridyl-2)-2-phenyl-4-methylpiperazine comprises the following steps:
(a) suspending a pyridinecarboxylate in an organic solvent; and
(b) adding an organoaluminum hydride to the reaction mixture of step (a) to produce 1-(3-hydroxymethylpyridyl-2)-2-phenyl-4-methylpiperazine.

2. The process according to claim 1 further comprising the steps of quenching the mixture of step (b) and isolating the 1-(3-hydroxymethylpyridyl-2)-2-phenyl-4-methylpiperazine from the quenched reaction mass.

3. The process according to claim 1 wherein the pyridinecarboxylate is a pyridinecarboxylic acid.

4. The process according to claim 1 wherein the organoaluminum hydride is sodium bis(2-methoxyethoxy)aluminum hydride.

5. The process according to claim 4 wherein the sodium bis(2-methoxyethoxy)aluminum hydride is added in a range of about 2 moles of sodium bis(2-methoxyethoxy)aluminum hydride per mole of pyridinecarboxylate to about 6 moles of sodium bis(2-methoxyethoxy)aluminum hydride per mole of pyridinecarboxylate.

6. The process according to claim 5 wherein the sodium bis(2-methoxyethoxy)aluminum hydride is added in a range of about 2.5 moles of sodium bis(2-methoxyethoxy)aluminum hydride per mole of pyridinecarboxylate to about 5 moles of sodium bis(2-methoxyethoxy)aluminum hydride per mole of pyridinecarboxylate.

7. The process according to claim 4 wherein the sodium bis(2-methoxyethoxy)aluminum hydride is added in a range of about 3 moles of sodium bis(2-methoxyethoxy)aluminum hydride per mole of pyridinecarboxylate to about 3.5 moles of sodium bis(2-methoxyethoxy)aluminum hydride per mole of pyridinecarboxylate.

8. The process according to claim 2 wherein the quenching step comprises adding an organic solvent and an aqueous solution of an alkali or alkali earth metal salt to the reaction mass of step (b).

9. The process according to claim 8 wherein the organic solvent is a C1 to C4 alcohol.

10. The process according to claim 8 wherein the aqueous solution is a sodium sulfate aqueous solution.

11. A process for preparing mirtazapine comprising:
(a) hydrolyzing 1-(3-cyanopyridyl-2)-4-methyl-2-phenylpiperazine to form 1-(3-carboxypyridyl-2)-4-methyl-2-phenylpiperazine;
(b) reducing the 1-(3-carboxypyridyl-2)-4-methyl-2-phenylpiperazine with an organoaluminum hydride to give 1-(3-hydroxymethyllpyridyl-2)-2-phenyl-4-methylpiperazine; and
(c) cyclizing 1-(3-hydroxymethyllpyridyl-2)-2-phenyl-4-methylpiperazine with a dehydrating agent to give mirtazapine.

12. The process of claim 11 further comprising the steps of quenching the mixture of step (b) and isolating the 1-(3-hydroxymethylpyridyl-2)-2-phenyl-4-methylpiperazine from the quenched reaction mass.

13. The process according to claim 11 wherein the organoaluminum hydride is sodium bis(2-methoxyethoxy)aluminum hydride.

14. The process according to claim 13 wherein the sodium bis(2-methoxyethoxy)aluminum hydride is added in a range of about 2 moles of sodium bis(2-methoxyethoxy)aluminum hydride per mole of 1-(3-carboxypyridyl-2)-4-methyl-2-phenylpiperazine to about 6 moles of sodium bis(2-methoxyethoxy)aluminum hydride per mole of 1-(3-carboxypyridyl-2)-4-methyl-2-phenylpiperazine.

15. The process according to claim 13 wherein the sodium bis(2-methoxyethoxy)aluminum hydride is added in a range of about 2.5 moles of sodium bis(2-methoxyethoxy)aluminum hydride per mole of 1-(3-carboxypyridyl-2)-4-methyl-2-phenylpiperazine to about 5 moles of sodium bis(2-methoxyethoxy)aluminum hydride per mole of 1-(3-carboxypyridyl-2)-4-methyl-2-phenylpiperazine.

16. The process according to claim 13 wherein the sodium bis(2-methoxyethoxy)aluminum hydride is added in a range of about 3 moles of sodium bis(2-methoxyethoxy)aluminum hydride per mole of 1-(3-carboxypyridyl-2)-4-methyl-2-phenylpiperazine to about 3.5 moles of sodium bis(2-methoxyethoxy)aluminum hydride per mole of 1-(3-carboxypyridyl-2)-4-methyl-2-phenylpiperazine.

17. The process according to claim 12 wherein the quenching step comprises adding an organic solvent and an aqueous solution of an alkali or alkali earth metal salt to the reaction mass of step (b).

18. The process according to claim 17 wherein the organic solvent is a C₁ to C₄ alcohol.

19. The process according to claim 17 wherein the aqueous solution is a sodium sulfate aqueous solution.
